(19) United States
(12) Patent Application Publication
(10) Pub. No.: US 2025/0262384 A1
(43) Pub. Date: Aug. 21, 2025

(54) Auto-Disabling Syringe Assembly

(71) Applicant: Thomas J. Shaw, Frisco, TX (US)

(72) Inventor: Thomas J. Shaw, Frisco, TX (US)

(21) Appl. No.: 19/051,863

(22) Filed: Feb. 12, 2025

Related U.S. Application Data
(60) Provisional application No. 63/554,329, filed on Feb. 16, 2024.

(51) Int. Cl. A61M 5/315

(52) U.S. Cl. CPC A61M 5/31505; A61M 2005/31508

(57) ABSTRACT

Systems and methods are disclosed that include providing a syringe with a barrel having a cylindrical body portion and a nose end, a needle assembly having a needle extending from the nose end of the barrel, and a plunger assembly having a plunger surface for selectively actuating the plunger assembly, an elongated plunger body extending therefrom, and a plunger seal that forms a fluid tight seal between the barrel and the plunger assembly. The elongated plunger body includes a ribbed portion, an annular clip channel disposed adjacent to the ribbed portion, and a disabling clip disposed around the elongated plunger body. The disabling clip allows withdrawal of the plunger assembly within the barrel and rides forward in the annular clip channel with compression of the plunger assembly to engage with the barrel to prevent withdrawal of the plunger assembly from the barrel after first use of the syringe.

### CROSS-REFERENCE TO RELATED APPLICATIONS

This application claims priority to U.S. Provisional Application No. 63/554,329, filed on Feb. 16, 2024, the disclosure of which is incorporated by reference in its entirety for all purposes.

### BACKGROUND OF THE INVENTION

Medical syringes are used for administering injections or infusions of drugs, medicines, and vaccines all over the world. In an effort to control the spread of blood-borne pathogens and illnesses and the incidence of contamination by contact with either exposed needles or bodily fluids, syringes have been designed to embody various safety features, such as retractable needles and needle covers. These features are useful in protecting medical personnel from accidental “sticks” when administering injections or infusions. Syringes have also been designed to embody disabling features that only allow a single use from a syringe to prevent the reuse or sharing of syringes. These features are common in self-administering syringes that are often used for injections and infusions of drugs, medicines, and vaccines by the person receiving the injection or infusion. These disabling features are embodied to prevent the reuse or sharing of syringes to further prevent the spread of blood-borne pathogens and illnesses between users that may share a syringe or by a user that may reuse a syringe that has become contaminated. However, in some instances, the disabling features may be breached or overridden in an attempt to enable reuse of a syringe, thereby allowing multiple uses from a single syringe and increasing the likelihood of spreading blood-borne pathogens and illnesses between users. Thus, the medical industry continues to demand advances in syringe technology to reduce and/or eliminate syringe abuse and increase safety for the user.

### SUMMARY

In some embodiments, the systems and methods described herein relate to a syringe, including: a barrel including a cylindrical body portion and a nose end; a needle assembly including a needle received within the needle assembly and extending from the nose end of the barrel; and a plunger assembly including a plunger surface for selectively actuating the plunger assembly, an elongated plunger body extending therefrom, and a plunger seal that forms a fluid tight seal between the cylindrical body portion of the barrel and the plunger assembly, wherein the elongated plunger body includes a ribbed portion, an annular clip stop, an annular clip channel disposed between the ribbed portion and the annular clip stop, and a disabling clip disposed around the elongated plunger body and configured to engage with the cylindrical body portion of the barrel to prevent withdrawal of the plunger assembly from the barrel after first use of the syringe.

In some embodiments, the systems and methods described herein related to a method of administering an injection, including: providing a syringe including: a barrel including a cylindrical body portion and a nose end; a needle assembly including a needle received within the needle assembly and extending from the nose end of the barrel; and a plunger assembly including a plunger surface for selectively actuating the plunger assembly, an elongated plunger body extending therefrom, and a plunger seal that forms a fluid tight seal between the cylindrical body portion of the barrel and the plunger assembly, wherein the elongated plunger body includes a ribbed portion, an annular clip stop, an annular clip channel disposed between the ribbed portion and the annular clip stop, and a disabling clip disposed around the elongated plunger body; drawing a medicine through the needle into the barrel by activation of the plunger assembly in a first direction; injecting the medicine from the barrel through the needle by activation of the plunger assembly in a second opposing direction; and preventing subsequent use of the syringe via engagement of the disabling clip with the cylindrical body portion of the barrel to prevent withdrawal of the plunger assembly from the barrel after first use of the syringe.

### BRIEF DESCRIPTION OF THE DRAWINGS

For a more complete understanding of the present disclosure and so that the manner in which the features and advantages of the embodiments can be understood in more detail, reference is now made to the following brief description, taken in connection with the accompanying drawings and detailed description.

FIG. 1 shows an orthogonal side view of a syringe according to an embodiment of the disclosure.

FIG. 2 shows an orthogonal cross-sectional side view of the syringe according to an embodiment of the disclosure.

FIG. 3 shows an oblique exploded view of the syringe according to an embodiment of the disclosure.

FIG. 4 shows an oblique view of a disabling clip of the syringe according to an embodiment of the disclosure.

FIG. 5 shows an orthogonal side view of the syringe prepared for use according to an embodiment of the disclosure.

FIG. 6 shows an orthogonal cross-sectional side view of the syringe prepared for use according to an embodiment of the disclosure.

FIG. 7 shows an orthogonal side view of the syringe after use according to an embodiment of the disclosure.

FIG. 8 shows an orthogonal cross-sectional side view of the syringe after use according to an embodiment of the disclosure.

FIG. 9 shows an orthogonal side view of a syringe according to another embodiment of the disclosure.

FIG. 10 shows an orthogonal cross-sectional side view of the syringe according to an embodiment of the disclosure.

FIG. 11 shows an oblique exploded view of the syringe according to an embodiment of the disclosure.

FIG. 12 shows an orthogonal side view of the syringe prepared for use according to an embodiment of the disclosure.

FIG. 13 shows an orthogonal cross-sectional side view of the syringe prepared for use according to an embodiment of the disclosure.

FIG. 14 shows an orthogonal side view of the syringe after use according to an embodiment of the disclosure.

FIG. 15 shows an orthogonal cross-sectional side view of the syringe after use according to an embodiment of the disclosure.

FIG. 16 shows a flowchart of a method of administering an injection with a syringe according to an embodiment of the disclosure.

The use of the same reference symbols in different drawings indicates similar or identical items.

### DETAILED DESCRIPTION

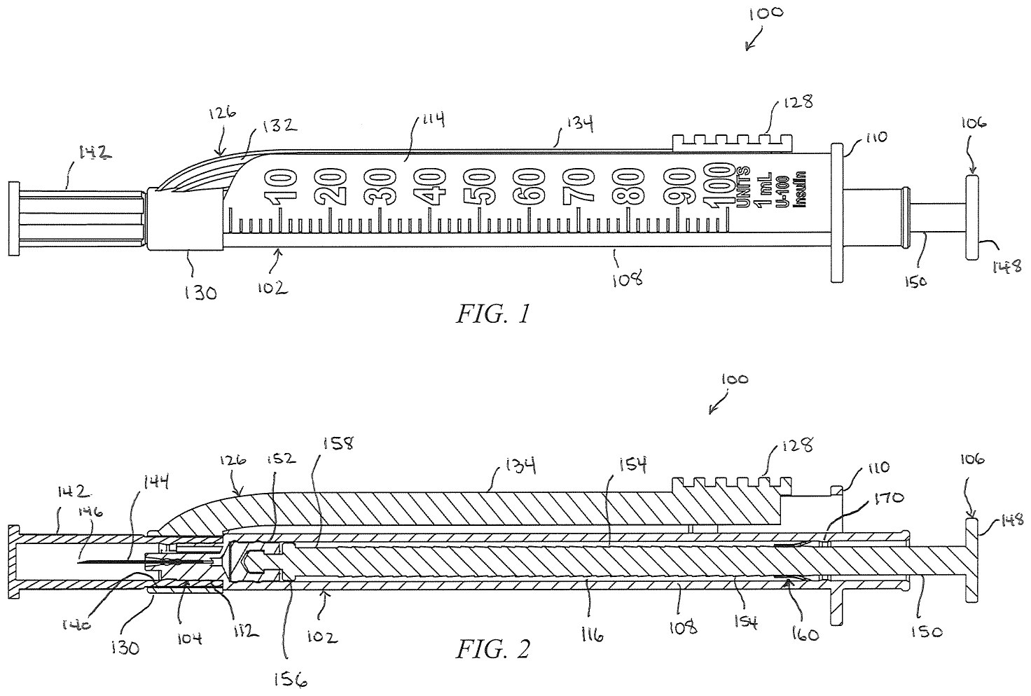

Referring to FIGS. 1 to 3, an orthogonal side view, an orthogonal cross-sectional side view, and an oblique exploded view of a syringe 100 are shown according to an embodiment of the disclosure. The syringe 100 may generally comprise a barrel 102, a needle assembly 104 coupled to the barrel 102, and a plunger assembly 106 disposed within the barrel 102.

Barrel 102 may comprise a cylindrical body portion 108 having one or more projecting finger flanges 110, a nose end 112 for receiving needle assembly 104, and a pair of opposing indicia display surfaces 114 that extends from the cylindrical body portion 108. The opposing indicia display surfaces 114 may generally be substantially flat and extend substantially tangentially from the cylindrical body portion 108. One or more of the opposing indicia display surfaces 114 may be marked with an easily readable volumetric scale comprising appropriate indicia adjacent to the cylindrical body portion 108 to facilitate easy reading of a liquid level inside an inner fluid chamber 116 of the cylindrical body portion 108 of the barrel 102. Each of the opposing indicia display surfaces 114 may comprise a longitudinally extending, inwardly projecting rail 122 provided on opposing inwardly facing walls 124 of each of the opposing indicia display surfaces 114.

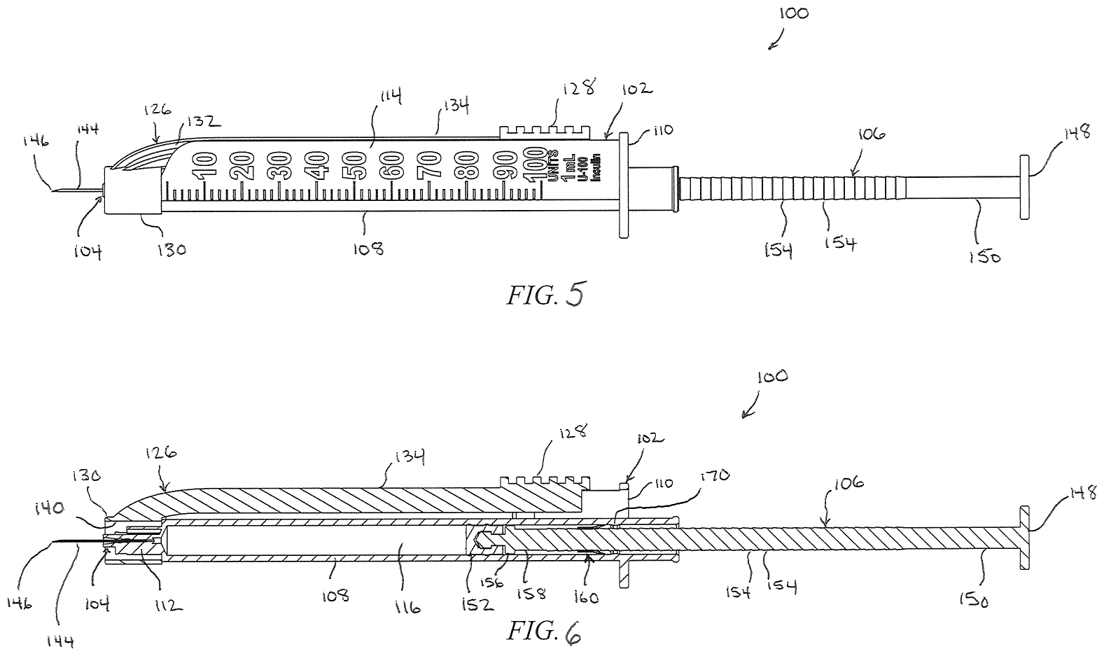

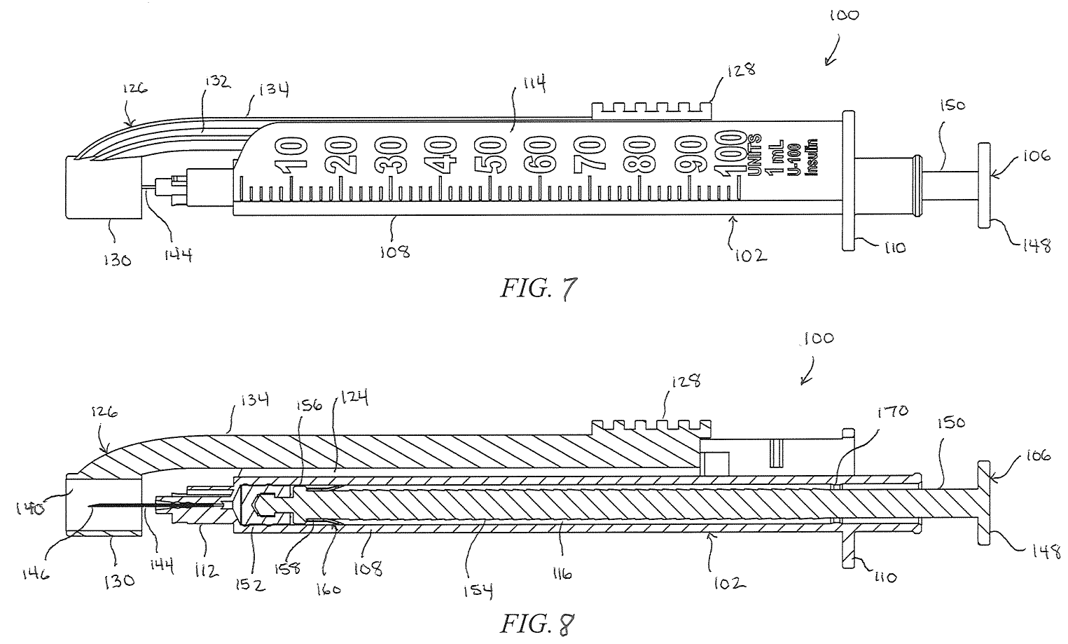

Barrel 102 may also comprise a needle safety device 126 unitarily molded from a polymeric material. The needle safety device 126 may comprise an activation surface 128 used to slide the needle safety device 126, a needle tip shield 130, and a pair of channels 132 disposed along a main body portion 134 of the needle safety device 126. The longitudinally extending, inwardly projecting rails 122 of the opposing indicia display surfaces 114 may be cooperatively sized and configured to engage and provide a smooth, slidable interface with the channels 132 of the needle safety device 126. Each of the opposing indicia display surfaces 114 may also comprise a rear stop 136 and a slide stop 138 disposed rearwardly of the longitudinally extending, inwardly projecting rails 122. The rear stops 136 may be configured to engage the channels 132 of the needle safety device 126 and retain the needle safety device 126 in a first retracted position (shown in FIGS. 5 and 6) prior to use of the syringe 100, and the slide stops 138 may be configured to engage the channels 132 of the needle safety device 126 and retain the needle safety device 126 in a second extended position (shown in FIGS. 7 and 8) after use of the syringe 100.

In some embodiments, needle tip shield 130 may comprise an inner bore 140 that is coaxially aligned with the nose end 112 of the barrel 102 and configured to slide over and surround the nose end 112 of the barrel 102. Further, in some embodiments, inner bore 140 of the needle tip shield 130 may receive and retain a needle cap 142. Barrel 102 is preferably molded from a medical grade polymeric material that is sufficiently transparent to permit the liquid level drawn into the inner fluid chamber 116 of the cylindrical body portion 108 of the barrel 102 to be plainly viewed by a user through one or more of the opposing indicia display surfaces 114 comprising the easily readable volumetric scale.

Needle assembly 104 may generally comprise a needle 144 received within and extending from the nose end 112 of the barrel 102. In some embodiments, needle assembly 104 may comprise a needle holder or hub (not shown) that retains the needle 144 within the nose end 112 of the barrel 102 and in fixed relation to the barrel 102 when the syringe 100 is fully assembled. As will be discussed later herein, the needle safety device 126 may be selectively positioned between the first retracted position (shown in FIGS. 5 and 6) to expose a needle tip 146 of the needle 144 for selective use of the syringe 100 and the second extended position (shown in FIGS. 7 and 8) to cover up the needle tip 146 of the needle 144 after use of the syringe 100 to protect a user or other from accidental “sticks” from an otherwise exposed needle of a traditional syringe not having the needle safety device 126.

Plunger assembly 106 may generally comprise a plunger surface 148 for selectively actuating the plunger assembly 106, an elongated plunger body 150 extending therefrom, and a plunger seal 152 disposed on or about a distal end of the elongated plunger body 150 of the plunger assembly 106. The elongated plunger body 150 may be at least partially received within the cylindrical body portion 108 of the barrel 102, such that the plunger seal 152 forms a fluid tight seal between plunger assembly 106 and the barrel 102, and more specifically, between the elongated plunger body 150 of the plunger assembly 106 and the cylindrical body portion 108 of the barrel 102.

The elongated plunger body 150 may comprise a series of beveled ribs 154 extending along a length of the elongated plunger body 150, an annular clip stop 156, and an annular clip channel 158 disposed therebetween. The beveled ribs 154 may generally comprise frustoconical segments that extend consecutively along the length of the elongated plunger body 150, such that truncated portions of each segment angle away from nose end 112 of barrel 102 and form ridges facing the nose end 112 of the barrel 102. This configuration orients beveled portions of each segment rearward towards the plunger surface 148 and away from the nose end 112 of the barrel 102. The annular clip stop 156 comprises an enlarged annular ridge disposed above the plunger seal 152. In some embodiments, the annular clip stop 156 may provide a rearward seating surface for the plunger seal 152. The annular clip channel 158 comprises a cylindrical portion disposed between the beveled rib 154 disposed closest to the distal end of the plunger assembly 106 and the annular clip stop 156.

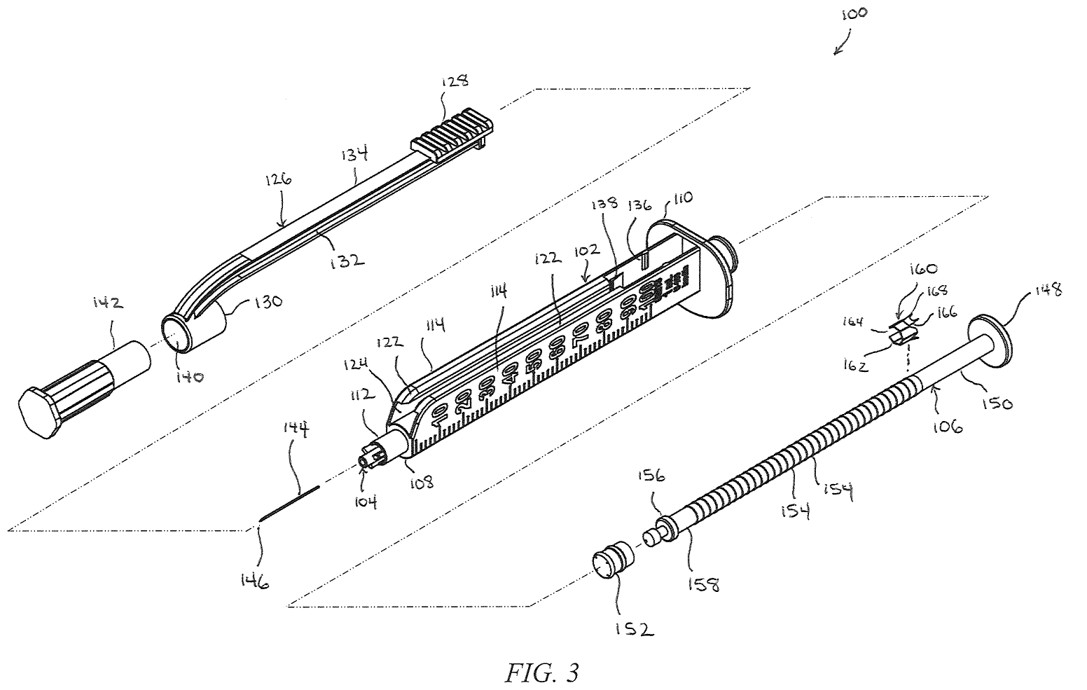

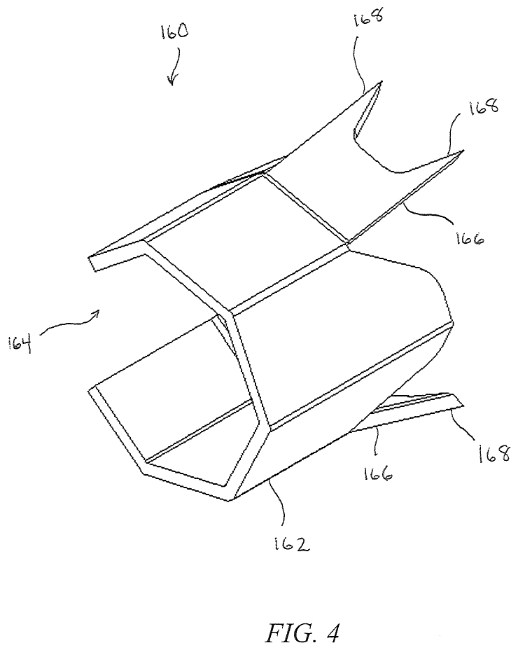

Referring FIGS. 3 and 4, plunger assembly 106 may also comprise a disabling clip 160 disposed around the elongated plunger body 150 of the plunger assembly 106. The disabling clip 160 may comprise a main annular body portion 162 that extends around the elongated plunger body 150 of the plunger assembly 106. In some embodiments, the main annular body portion 162 may comprise a notch 164 that provides the main annular body portion 162 a substantially C-shaped cross-sectional profile such that the main annular body portion 162 can be snapped onto the elongated plunger body 150 of the plunger assembly 106 and such that the main annular body portion 162 extends only partially around the elongated plunger body 150 of the plunger assembly 106. In some embodiments, the main annular body portion 162 may extend at least about 270 degrees around the elongated plunger body 150 of the plunger assembly 106. However, in other embodiments, the main annular body portion 162 may extend all the way around the elongated plunger body 150 of the plunger assembly 106.

Disabling clip 160 may also comprise a pair of opposing angled legs 166 extending angularly from the main annular body portion 162. In some embodiments, the disabling clip 160 may comprise two opposing angled legs 166. However, in other embodiments, the disabling clip 160 may comprise more than two angled legs 166. The angled legs 166 may extend from the main annular body portion 162 at an angle of at least 15 degrees, or even at least 30 degrees. Each angled leg 166 may comprise one or more barrel tangs 168 extending from the end of the angled leg 166. The disabling clip 160 may be formed from a corrosion-resistant metal, such as nickel, stainless steel, titanium, and any alloy thereof. In some embodiments, the one or more metallic barrel tangs 168 may be configured to engage and/or “bite into” the polymeric cylindrical body portion 108 of the barrel 102 to prevent the plunger assembly 106 from being withdrawn from the barrel 102 after the first use of the syringe 100, thereby automatically disabling the syringe 100 after its first use.

Referring to FIGS. 5 and 6, an orthogonal side view and an orthogonal cross-sectional side view of the syringe 100 prepared for use are shown according to an embodiment of the disclosure. With the needle safety device 126 disposed in the first retracted position, the needle cap 142 may be removed from the syringe 100 to prepare the syringe 100 for use. To use the syringe 100, the needle 144 may be inserted through the stopper of a medicine vial (e.g., an insulin bottle), and the medicine may be drawn through the needle 144 and into the inner fluid chamber 116 of the cylindrical body portion 108 of the barrel 102 by pulling rearwardly on the plunger surface 148 of the plunger assembly 106.

During withdrawal of the plunger assembly 106, and due to the rearward-facing bevels of the beveled ribs 154 of the plunger assembly 106, the beveled ribs 154 on the elongated plunger body 150 may slide through the disabling clip 160 with minimal restriction, while the disabling clip 160 may be held longitudinally stationary by an annular stop ridge 170 disposed at a rearward portion of the cylindrical body portion 108 of the barrel 102. As shown in FIGS. 5 and 6, plunger assembly 106 is withdrawn through the barrel 102 during medicinal withdrawal from the medicine vial. When the plunger assembly 106 is fully withdrawn within the cylindrical body portion 108 of the barrel 102, the disabling clip 160 may become disposed within and/or lock into the annular clip channel 158, which is disposed forwardly of the beveled ribs 154 and rearwardly of the annular clip stop 156. The disabling clip 160 may be retained within the annular clip channel 158 and will ride forward in the annular clip channel 158 with the plunger assembly 106 as the plunger surface 148 of the plunger assembly 106 is pressed forwardly to administer the injection with the syringe 100.

Referring to FIGS. 7 and 8, an orthogonal side view and an orthogonal cross-sectional side view of the syringe 100 after use are shown according to an embodiment of the disclosure. After administration of the injection with the syringe 100, the plunger seal 152 may reach the end of the cylindrical body portion 108 of the barrel 102, and most, if not all, of the medicine will have discharged from the inner fluid chamber 116 of the cylindrical body portion 108 of the barrel 102 through the needle 144. The barrel tangs 168 of the disabling clip 160 will interact with the cylindrical body portion 108 of the barrel 102 in response to any attempted withdrawal of the plunger assembly 106 from the barrel 102. More specifically, the metallic barrel tangs 168 may engage or bite into the polymeric cylindrical body portion 108 of the barrel 102 and prevent the plunger assembly 106 from being withdrawn from the barrel 102 after use of the syringe 100. This automatically disables the syringe 100 after its first use and prevents subsequent use of the syringe 100.

Following an injection, needle safety device 126 may be activated forwardly by pressing on the activation surface 128. As the needle safety device 126 slides forwardly, the slide stops 138 may engage the channels 132 of the needle safety device 126 and lock the needle safety device 126 in the second extended position. In the second extended position, the needle tip 146 may be disposed within the inner bore 140 of the needle tip shield 130, thereby circumferentially surrounding the needle tip 146 and exposed portions of the needle 144. Once engaged, the slide stops 138 effectively prevent the needle safety device 126 from being slid backwards to uncover the needle tip 146 and/or exposed portions of the needle 144, thereby preventing unintended needle sticks or inadvertent contact with the needle 144.

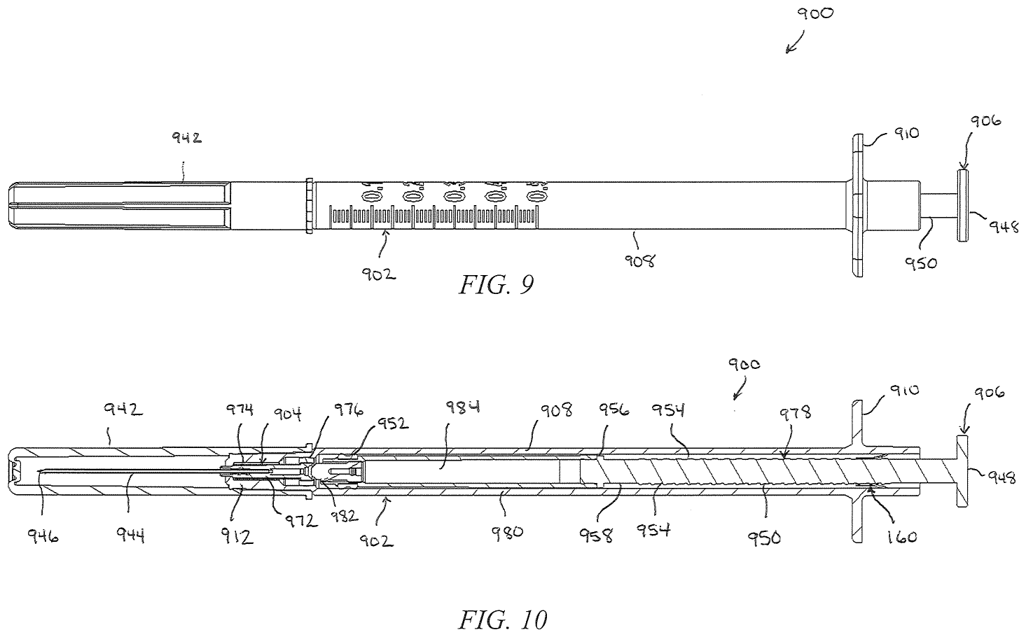

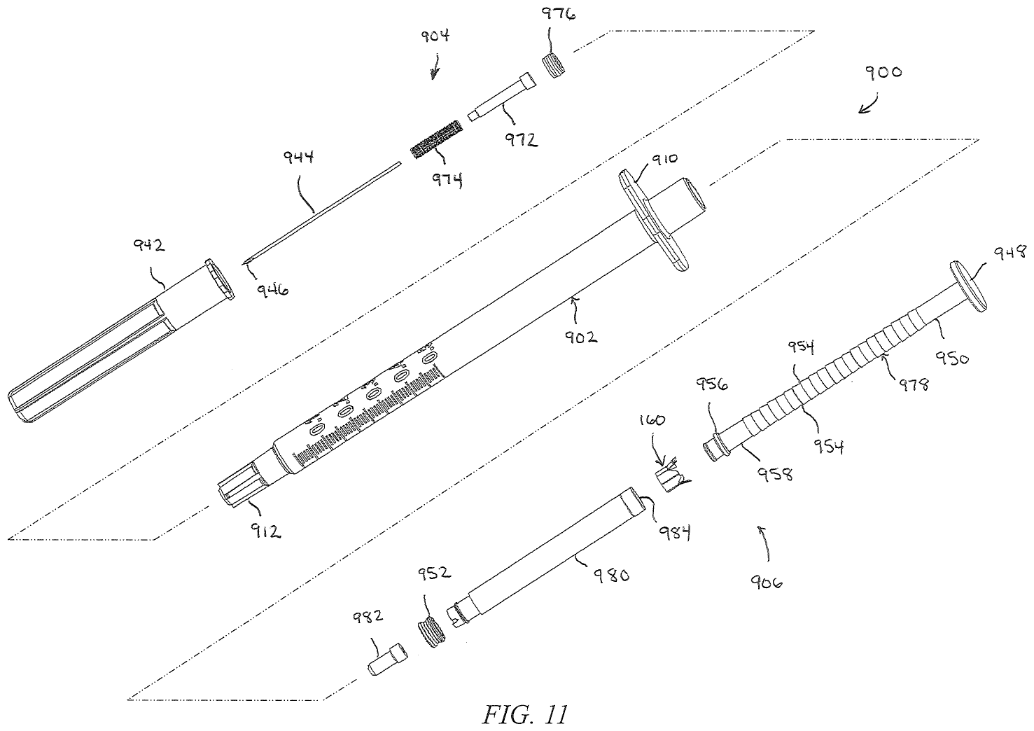

Referring now to FIGS. 9 to 11, an orthogonal side view, an orthogonal cross-sectional side view, and an oblique exploded view of a syringe 900 are shown according to another embodiment of the disclosure. The syringe 900 may generally comprise a barrel 902, a needle assembly 904 coupled to the barrel 902, and a plunger assembly 906 disposed within the barrel 902. Barrel 902 may comprise a cylindrical body portion 908 having one or more projecting finger flanges 910 and a nose end 912 for receiving needle assembly 904. In some embodiments, barrel 902 may be marked with an easily readable volumetric scale comprising appropriate indicia adjacent to the cylindrical body portion 908. Barrel 102 may preferably be molded from a medical grade polymeric material that is sufficiently transparent to permit the liquid level drawn into an inner fluid chamber 916 of the cylindrical body portion 108 of the barrel 102 to be plainly viewed by a user through one or more of the opposing indicia display surfaces 114 comprising the easily readable volumetric scale. In some embodiments, syringe 900 may also comprise a selectively removable needle cap 942 that slides over and engages the nose end 912 of the barrel 902 prior to use.

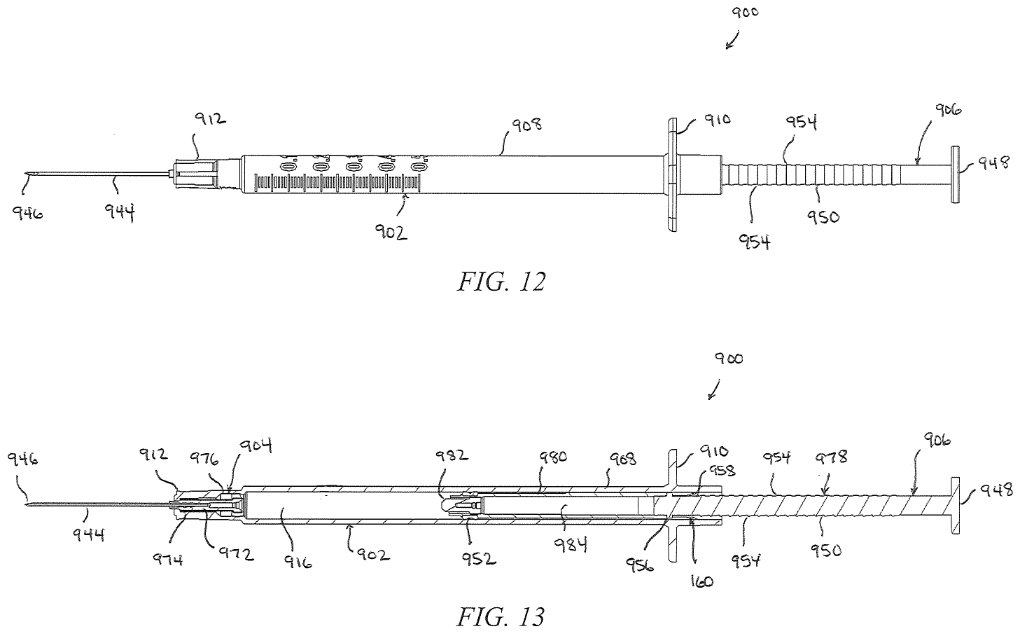

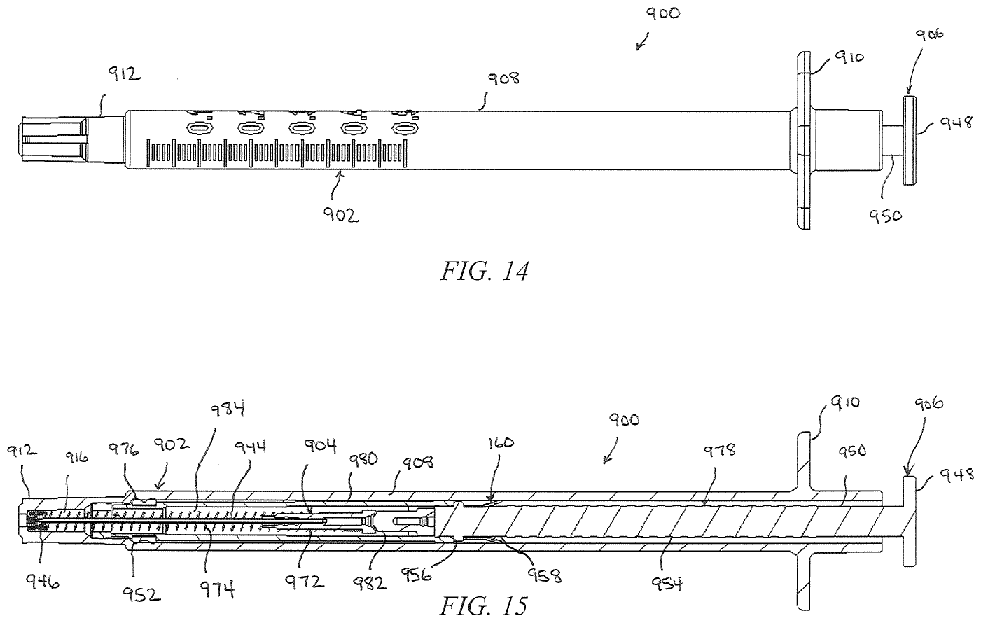

Needle assembly 904 may generally be received within and extending from the nose end 912 of the barrel 902. Needle assembly 904 may comprise a needle holder 972 that retains a needle 944 and that is biased against the nose end 912 of the barrel 902 by a compression spring 974. When the syringe 900 is fully assembled and ready for use, an elastomeric seal 976 may be disposed between a head section of the needle holder 972 and the barrel 902 to hold the needle assembly 904 in fixed relation to the barrel 902 prior to retraction of the needle assembly 904. As will be discussed later herein, the needle assembly 904 may be selectively retracted between a first use position (shown in FIGS. 12 and 13) to expose a needle tip 946 of the needle 944 for selective use of the syringe 100 and a second retracted position (shown in FIGS. 14 and 15) where the needle assembly 904 is retracted within the barrel 902 after use of the syringe 900 to protect a user or other from accidental “sticks” from an otherwise exposed needle and prevent subsequent use of the syringe 900.

Plunger assembly 906 may generally comprise a main plunger body 978 comprising a plunger surface 948 for selectively actuating the plunger assembly 906 and an elongated plunger body 950 extending therefrom. Plunger assembly 906 may also comprise a needle retraction actuator 980 that engages with an end of the elongated plunger body 950 of the main plunger body 978. A plunger seal 952 may be disposed on or about a distal end of the needle retraction actuator 980 of the plunger assembly 906. In some embodiments, needle retraction actuator 980 may also comprise a plunger tip 982 that functions to maximize the fluid dispersed from the inner fluid chamber 916 of the barrel 902 during use. The plunger assembly 906 may be at least partially received within the cylindrical body portion 908 of the barrel 902, such that the plunger seal 952 forms a fluid tight seal between plunger assembly 906 and the barrel 902, and more specifically, between the needle retraction actuator 980 of the plunger assembly 906 and the inner fluid chamber 916 of the cylindrical body portion 908 of the barrel 902.

The elongated plunger body 950 of the main plunger body 978 may comprise a series of beveled ribs 954 extending along a length of the elongated plunger body 950, an annular clip stop 956, and an annular clip channel 958 disposed therebetween. The beveled ribs 954 may generally comprise frustoconical segments that extend consecutively along the length of the elongated plunger body 950, such that truncated portions of each segment angle away from nose end 912 of barrel 9102 and form ridges facing the nose end 912 of the barrel 902. This configuration orients beveled portions of each segment rearward towards the plunger surface 948 and away from the nose end 912 of the barrel 902. The annular clip stop 956 comprises an enlarged annular ridge disposed above the plunger seal 952. In some embodiments, the annular clip stop 956 may provide a rearward seating surface for the needle retraction actuator 980, such that forward motion of the main plunger body 978 also moves the needle retraction actuator 980 forwardly with respect to the barrel 902. The annular clip channel 958 comprises a cylindrical portion disposed between the beveled rib 954 disposed closest to the distal end of the plunger assembly 906 and the annular clip stop 956.

Referring FIGS. 4 and 11, plunger assembly 906 may also comprise a disabling clip 160 disposed around the elongated plunger body 950 of the main plunger body 978 of the plunger assembly 906. The disabling clip 160 may comprise a main annular body portion 162 that extends around the elongated plunger body 950 of the main plunger body 978 of the plunger assembly 106. In some embodiments, the main annular body portion 162 may comprise a notch 164 that provides the main annular body portion 162 a substantially C-shaped cross-sectional profile such that the main annular body portion 162 can be snapped onto the elongated plunger body 950 of the main plunger body 978 of the plunger assembly 906 and such that the main annular body portion 162 extends only partially around the elongated plunger body 950 of the main plunger body 978 of the plunger assembly 906. In some embodiments, the main annular body portion 162 may extend at least about 270 degrees around the elongated plunger body 950 of the main plunger body 978 of the plunger assembly 906. However, in other embodiments, the main annular body portion 162 may extend all the way around the elongated plunger body 950 of the main plunger body 978 of the plunger assembly 906.

Disabling clip 160 may also comprise a pair of opposing angled legs 166 extending angularly from the main annular body portion 162. In some embodiments, the disabling clip 160 may comprise two opposing angled legs 166. However, in other embodiments, the disabling clip 160 may comprise more than two angled legs 166. The angled legs 166 may extend from the main annular body portion 162 at an angle of at least 15 degrees, or even at least 30 degrees. Each angled leg 166 may comprise one or more barrel tangs 168 extending from the end of the angled leg 166. The disabling clip 160 may be formed from a corrosion-resistant metal, such as nickel, stainless steel, titanium, and any alloy thereof. In some embodiments, the one or more metallic barrel tangs 168 may be configured to engage and/or “bite into” the polymeric cylindrical body portion 908 of the barrel 902 to prevent the plunger assembly 906 from being withdrawn from the barrel 902 after the first use of the syringe 900, thereby automatically disabling the syringe 900 after its first use.

Referring to FIGS. 12 and 13, an orthogonal side view and an orthogonal cross-sectional side view of the syringe 900 prepared for use are shown according to an embodiment of the disclosure. With the needle assembly 904 disposed in the first use position, the needle cap 942 may be removed from the syringe 900 to prepare the syringe 900 for use. To use the syringe 900, the needle 944 may be inserted through the stopper of a medicine vial (e.g., an insulin bottle), and the medicine may be drawn through the needle 944 and into the inner fluid chamber 916 of the cylindrical body portion 908 of the barrel 902 by pulling rearwardly on the plunger surface 948 of the plunger assembly 906.

During withdrawal of the plunger assembly 906, and due to the rearward-facing bevels of the beveled ribs 954 of the plunger assembly 906, the beveled ribs 954 on the elongated plunger body 950 of the main plunger body 978 may slide through the disabling clip 160 with minimal restriction, while the disabling clip 160 may be held longitudinally stationary at a rearward portion of the cylindrical body portion 908 of the barrel 902. As shown in FIGS. 12 and 13, main plunger body 978 of the plunger assembly 906 is withdrawn through the barrel 902 during medicinal withdrawal from the medicine vial. When the plunger assembly 906 is fully withdrawn within the cylindrical body portion 908 of the barrel 902, the disabling clip 160 may become disposed within and/or lock into the annular clip channel 958, which is disposed forwardly of the beveled ribs 954 and rearwardly of the annular clip stop 956. The disabling clip 160 may be retained within the annular clip channel 958 and will ride forward in the annular clip channel 958 with the plunger assembly 906 as the plunger surface 948 of the main plunger body 978 of the plunger assembly 906 is pressed forwardly to administer the injection with the syringe 900.

Referring to FIGS. 14 and 15, an orthogonal side view and an orthogonal cross-sectional side view of the syringe 900 after use are shown according to an embodiment of the disclosure. After administration of the injection with the syringe 900, the plunger seal 952 may reach the end of the cylindrical body portion 908 of the barrel 902, and most, if not all, of the medicine will have discharged from the inner fluid chamber 916 of the cylindrical body portion 908 of the barrel 902 through the needle 944. The barrel tangs 168 of the disabling clip 160 will interact with the cylindrical body portion 908 of the barrel 902 in response to any attempted withdrawal of the plunger assembly 906 from the barrel 02. More specifically, the metallic barrel tangs 168 may engage or bite into the polymeric cylindrical body portion 908 of the barrel 902 and prevent the plunger assembly 906 from being withdrawn from the barrel 902 after use of the syringe 900. This automatically disables the syringe 900 after its first use and prevents subsequent use of the syringe 900.

During administration of the injection, needle retraction actuator 980 may be driven forwardly with the forward motion of the main plunger body 978 through the engagement of the needle retraction actuator 980 and the annular clip stop 956 of the main plunger body 978. Needle retraction actuator 980 may contact the elastomeric seal 976 as the plunger assembly 906 nears complete forward advancement within the barrel 902. Needle retraction actuator 980 may drive the elastomeric seal 976 forward, thereby disengaging it from the needle holder 972. Without restriction from the friction force the elastomeric seal 976 provides between the needle holder 972 and the barrel 902, the compression spring 974 that rearwardly biases the needle holder 972 against the nose end 912 of the barrel 902 may expand, thereby driving the needle assembly 904 rearward into an inner cavity 984 of the needle retraction actuator 980. Retraction of the needle assembly 904 is complete when the needle tip 946 of the needle 944 is withdrawn into the nose end 912 of the barrel 902 of the syringe 900, thereby preventing unintended needle sticks or inadvertent contact with the needle 944 after administration of the injection.

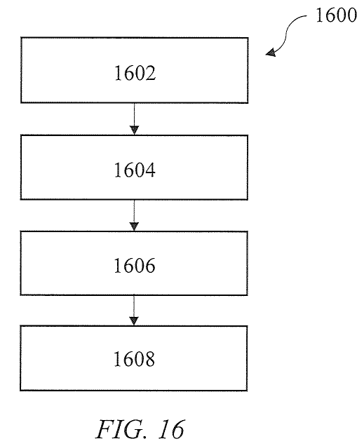

FIG. 16 shows a flowchart of a method 1600 of administering an injection with a syringe 100, 900 according to an embodiment of the disclosure. The method 1600 may begin at block 1602 by providing a syringe 100, 900 as disclosed herein. In some embodiments, the syringe 100, 900 may comprise a barrel 102, 902, a needle assembly 104, 904 comprising a needle 144, 944 extending from the barrel 102, 902, and a plunger assembly 106, 906 at least partially disposed within the barrel 102, 902 and comprising a plunger surface 148, 948 for selectively actuating the plunger assembly 106, 906, an elongated plunger body 150, 950 extending therefrom, and a plunger seal 152, 952. In some embodiments, the elongated plunger body 150, 950 may comprise a series of beveled ribs 154, 954 extending along a length of the elongated plunger body 150, 950, an annular clip stop 156, 956, an annular clip channel 158, 958 disposed therebetween, and a disabling clip 160 disposed at least partially around the elongated plunger body 150, 950.

The method 1600 may continue at block 1604 by drawing a medicine through the needle 144, 944 into the barrel 102, 902 by activation of the plunger assembly 106, 906 in a first direction. In some embodiments, activation of the plunger assembly 106, 906 in the first direction may cause the series of beveled ribs 154, 954 extending along a length of the elongated plunger body 150, 950 to slide through the disabling clip 160 while the disabling clip 160 is retained at a rearward portion of a cylindrical body portion 108, 908 of the barrel 102, 902. In some embodiments, activation of the plunger assembly 106, 906 in the first direction may cause the disabling clip 160 to be disposed within and/or lock into the annular clip channel 158, 958.

The method 1600 may continue at block 1606 by injecting the medicine from the barrel 102, 902 through the needle 144, 944 by activation of the plunger assembly 106, 906 in a second opposing direction. In some embodiments, activation of the plunger assembly 106, 906 in the second direction may cause the disabling clip 160 to ride forward in the annular clip channel 158, 958 with the plunger assembly 106, 906.

The method 1600 may continue at block 1608 by preventing subsequent use of the syringe 100, 900. In some embodiments, preventing subsequent use of the syringe 100, 900 may be accomplished by the disabling clip 160 preventing withdrawal of the plunger assembly 106, 906 from the barrel 102, 902. More specifically, in some embodiments, preventing subsequent use of the syringe 100, 900 may be accomplished by the barrel tangs 168 engaging or biting into the cylindrical body portion 108, 908 of the barrel 102, 902. In some embodiments, preventing subsequent use of the syringe 100, 900 may be accomplished by sliding a needle safety device 126 from a first rearward position to a second forward position to cause a needle tip shield 130 of the needle safety device 126 to circumferentially surround a needle tip 146 of the needle 144 and/or exposed portions of the needle 144 to prevent unintended needle sticks or inadvertent contact with the needle 144. In some embodiments, preventing subsequent use of the syringe 900 may be accomplished by automatic retraction of the needle assembly 904 into the barrel 902 of the syringe 900. In some embodiments, automatic retraction of the needle assembly 904 may be accomplished by a needle retraction actuator 980 contacting an elastomeric seal 976 disposed between the needle holder 972 and the barrel 902 to disengage the elastomeric seal 976 from the needle holder 972 to allow a compression spring 974 that rearwardly biases the needle holder 972 against the nose end 912 of the barrel 902 to expand and drive the needle assembly 904 rearward into an inner cavity 984 of the needle retraction actuator 980. Retraction of the needle assembly 904 is complete when the needle tip 946 of the needle 944 is withdrawn into the nose end 912 of the barrel 902 of the syringe 900.

Embodiments of a syringe 100, 900 and/or method 1600 of administering an injection with a syringe 100, 900 may include one or more of the following embodiments:

Embodiment 1. A syringe, comprising: a barrel comprising a cylindrical body portion and a nose end; a needle assembly comprising a needle received within the needle assembly and extending from the nose end of the barrel; and a plunger assembly comprising a plunger surface for selectively actuating the plunger assembly, an elongated plunger body extending therefrom, and a plunger seal that forms a fluid tight seal between the cylindrical body portion of the barrel and the plunger assembly, wherein the elongated plunger body comprises a ribbed portion, an annular clip channel disposed forwardly of the ribbed portion, and a disabling clip disposed around the elongated plunger body and configured to engage with the cylindrical body portion of the barrel to prevent withdrawal of the plunger assembly from the barrel after first use of the syringe.

Embodiment 2. The syringe of embodiment 1, wherein the ribbed portion of the elongated plunger body comprises a series of beveled ribs extending along a length of the elongated plunger body of the plunger assembly.

Embodiment 3. The syringe of embodiment 2, wherein a beveled portion of each of the beveled ribs is angled rearwardly towards the plunger surface of the plunger assembly.

Embodiment 4. The syringe of embodiment 1, wherein an annular clip stop provides a rearward seating surface for the plunger seal, and wherein the annular clip channel is disposed between the ribbed portion and the annular clip stop.

Embodiment 5. The syringe of embodiment 1, wherein the disabling clip comprises a main annular body portion disposed around the elongated plunger body and a notch that provides the main annular body portion with a substantially C-shaped cross-sectional profile to enable the main annular body portion of the disabling clip to be laterally pressed onto the elongated plunger body of the plunger assembly.

Embodiment 6. The syringe of embodiment 5, wherein the notch comprises a width less than a diameter of the elongated plunger body.

Embodiment 7. The syringe of embodiment 5, wherein the disabling clip comprises a plurality of angled legs extending angularly from the main annular body portion of the disabling clip.

Embodiment 8. The syringe of embodiment 7, wherein each of the plurality of angled legs comprises one or more barrel tangs extending from an end of each of the plurality of angled legs.

Embodiment 9. The syringe of embodiment 1, wherein the disabling clip is formed from a corrosion-resistant metal, such as nickel, stainless steel, titanium, and any alloy thereof.

Embodiment 10. The syringe of embodiment 1, wherein the disabling clip is configured to allow the ribbed portion of the elongated plunger body to slide through the disabling clip while the disabling clip is held longitudinally stationary by an annular stop ridge disposed at a rearward portion of the cylindrical body portion of the barrel during withdrawal of the plunger assembly within the barrel to draw a medicine or drug into the cylindrical body portion of the barrel.

Embodiment 11. The syringe of embodiment 10, wherein the disabling clip is configured to lock into the annular clip channel of the elongated plunger body of the plunger assembly when the plunger assembly is fully withdrawn within the barrel.

Embodiment 12. The syringe of embodiment 11, wherein the disabling clip is retained within the annular clip channel and rides forward in the annular clip channel when the plunger assembly is advanced within the barrel to administer an injection with the syringe.

Embodiment 13. The syringe of embodiment 12, wherein one or more barrel tangs extending from the disabling clip engage with the cylindrical body portion of the barrel to prevent withdrawal of the plunger assembly from the barrel after the first use of the syringe.

Embodiment 14. The syringe of embodiment 13, wherein the engagement between the one or more barrel tangs and the cylindrical body portion of the barrel automatically disables subsequent use of the syringe after the first use of the syringe.

Embodiment 15. The syringe of embodiment 1, wherein the barrel comprises a pair of opposing indicia display surfaces that extend substantially tangentially from the cylindrical body portion of the barrel.

Embodiment 16. The syringe of embodiment 15, wherein one or more of the opposing indicia display surfaces are marked with an easily readable volumetric scale comprising indicia adjacent to the cylindrical body portion to facilitate easy reading of a liquid level inside the cylindrical body portion.

Embodiment 17. The syringe of embodiment 16, wherein each of the opposing indicia display surfaces comprise a longitudinally extending, inwardly projecting rail disposed on opposing inwardly facing walls of each of the opposing indicia display surfaces.

Embodiment 18. The syringe of embodiment 17, further comprising: a needle safety device comprising an activation surface used to slide the needle safety device, a needle tip shield, and a pair of channels disposed along a main body portion of the needle safety device.

Embodiment 19. The syringe of embodiment 18, wherein longitudinally extending, inwardly projecting rails disposed on opposing inwardly facing walls of each of the opposing indicia display surfaces are cooperatively sized and configured to engage and provide a smooth, slidable interface with the pair of channels of the needle safety device between a first retracted position prior that exposes a needle tip of the needle for selective use of the syringe and a second extended position to cover up the needle tip of the needle after the first use of the syringe.

Embodiment 20. The syringe of embodiment 19, wherein the barrel comprises a pair of slide stops that engage the pair of channels of the needle safety device and lock the needle safety device in the second extended position to prevent unintended needle sticks or inadvertent contact with the needle after the first use of the syringe.

Embodiment 21. The syringe of embodiment 1, wherein the needle assembly comprises a needle holder that retains the needle, and wherein the needle assembly is rearwardly biased against the nose end of the barrel by a compression spring.

Embodiment 22. The syringe of embodiment 21, wherein an elastomeric seal is disposed between a head section of the needle holder and the barrel to hold the needle assembly in fixed relation to the barrel prior to retraction of the needle assembly.

Embodiment 23. The syringe of embodiment 22, wherein the plunger assembly comprises a main plunger body comprising the plunger surface for selectively actuating the plunger assembly and the elongated plunger body extending therefrom.

Embodiment 24. The syringe of embodiment 23, wherein the plunger assembly comprises a needle retraction actuator that engages with the elongated plunger body the main plunger body, and wherein the needle retraction actuator is driven forwardly with forward motion of the main plunger body.

Embodiment 25. The syringe of embodiment 24, wherein the needle retraction actuator comprises a plunger seal.

Embodiment 26. The syringe of embodiment 24, wherein the needle retraction actuator comprises a plunger tip configured to maximize fluid dispersed from the barrel during administration of an injection.

Embodiment 27. The syringe of embodiment 24, wherein the needle retraction actuator is configured to contact and move the elastomeric seal forward in the barrel in response to activation of the plunger assembly to disengage the elastomeric seal from the needle holder to initiate retraction of the needle assembly into the barrel.

Embodiment 28. The syringe of embodiment 27, wherein the needle assembly retracts rearwardly into an inner cavity of the needle retraction actuator.

Embodiment 29. A method of administering an injection, comprising: providing a syringe comprising: a barrel comprising a cylindrical body portion and a nose end; a needle assembly comprising a needle received within the needle assembly and extending from the nose end of the barrel; and a plunger assembly comprising a plunger surface for selectively actuating the plunger assembly, an elongated plunger body extending therefrom, and a plunger seal that forms a fluid tight seal between the cylindrical body portion of the barrel and the plunger assembly, wherein the elongated plunger body comprises a ribbed portion, an annular clip channel disposed forwardly of the ribbed portion, and a disabling clip disposed around the elongated plunger body; drawing a medicine through the needle into the barrel by activation of the plunger assembly in a first direction; injecting the medicine from the barrel through the needle by activation of the plunger assembly in a second opposing direction; and preventing subsequent use of the syringe via engagement of the disabling clip with the cylindrical body portion of the barrel to prevent withdrawal of the plunger assembly from the barrel after first use of the syringe.

Embodiment 30. The method of embodiment 29, wherein the ribbed portion of the elongated plunger body comprises a series of beveled ribs extending along a length of the elongated plunger body of the plunger assembly.

Embodiment 31. The method of embodiment 30, wherein a beveled portion of each of the beveled ribs is angled rearwardly towards the plunger surface of the plunger assembly.

Embodiment 32. The method of embodiment 29, wherein the annular clip stop provides a rearward seating surface for the plunger seal.

Embodiment 33. The method of embodiment 29, wherein the disabling clip comprises a main annular body portion disposed around the elongated plunger body and a notch that provides the main annular body portion with a substantially C-shaped cross-sectional profile to enable the main annular body portion of the disabling clip to be laterally pressed onto the elongated plunger body of the plunger assembly.

Embodiment 34. The method of embodiment 33, wherein the notch comprises a width less than a diameter of the elongated plunger body.

Embodiment 35. The method of embodiment 33, wherein the disabling clip comprises a plurality of angled legs extending angularly from the main annular body portion of the disabling clip.

Embodiment 36. The method of embodiment 35, wherein each of the plurality of angled legs comprises one or more barrel tangs extending from an end of each of the plurality of angled legs.

Embodiment 37. The method of embodiment 29, wherein the disabling clip is formed from a corrosion-resistant metal, such as nickel, stainless steel, titanium, and any alloy thereof.

Embodiment 38. The method of embodiment 29, further comprising: sliding the ribbed portion of the elongated plunger body through the disabling clip while the disabling clip is held longitudinally stationary by an annular stop ridge disposed at a rearward portion of the cylindrical body portion of the barrel during withdrawal of the plunger assembly within the barrel in the first direction to draw a medicine or drug into the cylindrical body portion of the barrel.

Embodiment 39. The method of embodiment 38, further comprising: locking the disabling clip into the annular clip channel of the elongated plunger body of the plunger assembly when the plunger assembly is fully withdrawn within the barrel.

Embodiment 40. The method of embodiment 39, further comprising: retaining the disabling clip is within the annular clip channel when the plunger assembly is advanced within the barrel in the second opposing direction to administer an injection with the syringe.

Embodiment 41. The method of embodiment 40, further comprising: preventing withdrawal of the plunger assembly from the barrel in the first direction after the first use of the syringe through engagement of one or more barrel tangs extending from the disabling clip with the cylindrical body portion of the barrel.

Embodiment 42. The method of embodiment 41, further comprising: automatically disabling subsequent use of the syringe after the first use of the syringe through engagement between the one or more barrel tangs and the cylindrical body portion of the barrel.

Embodiment 43. The method of embodiment 29, wherein the barrel comprises a pair of opposing indicia display surfaces that extends substantially tangentially from the cylindrical body portion of the barrel.

Embodiment 44. The method of embodiment 43, wherein one or more of the opposing indicia display surfaces are marked with an easily readable volumetric scale comprising indicia adjacent to the cylindrical body portion to facilitate easy reading of a liquid level inside the cylindrical body portion.

Embodiment 45. The method of embodiment 44, wherein each of the opposing indicia display surfaces comprises a longitudinally extending, inwardly projecting rail disposed on opposing inwardly facing walls of each of the opposing indicia display surfaces.

Embodiment 46. The method of embodiment 45, wherein longitudinally extending, inwardly projecting rails are cooperatively sized and configured to engage and provide a smooth, slidable interface with the pair of channels of a needle safety device between a first retracted position prior that exposes a needle tip of the needle for selective use of the syringe and a second extended position to cover up the needle tip of the needle after the first use of the syringe.

Embodiment 47. The method of embodiment 46, further comprising: locking the needle safety device in the second extended position to prevent unintended needle sticks or inadvertent contact with the needle with a pair of slide stops that engage the pair of channels of the needle safety device after the first use of the syringe.

Embodiment 48. The method of embodiment 29, wherein the needle assembly comprises a needle holder that retains the needle, and wherein the needle assembly is rearwardly biased against the nose end of the barrel by a compression spring.

Embodiment 49. The method of embodiment 48, wherein an elastomeric seal is disposed between a head section of the needle holder and the barrel to hold the needle assembly in fixed relation to the barrel prior to retraction of the needle assembly.

Embodiment 50. The method of embodiment 49, wherein the plunger assembly comprises a main plunger body comprising the plunger surface for selectively actuating the plunger assembly and the elongated plunger body extending therefrom.

Embodiment 51. The method of embodiment 50, wherein the plunger assembly comprises a needle retraction actuator that engages with the elongated plunger body the main plunger body, and wherein the needle retraction actuator is driven forwardly with forward motion of the main plunger body.

Embodiment 52. The method of embodiment 51, wherein the needle retraction actuator comprises a plunger seal.

Embodiment 53. The method of embodiment 51, wherein the needle retraction actuator comprises a plunger tip configured to maximize fluid dispersed from the barrel during administration of an injection.

Embodiment 54. The method of embodiment 51, wherein the activation of the plunger assembly in the second opposing direction causes the needle retraction actuator to contact and move the elastomeric seal forward in the barrel to disengage the elastomeric seal from the needle holder to initiate automatic retraction of the needle assembly into the barrel.

Embodiment 55. The method of embodiment 54, wherein the needle assembly retracts rearwardly into an inner cavity of the needle retraction actuator.

In the foregoing specification, the concepts have been described with reference to specific embodiments. However, those of ordinary skill in the art appreciate that various modifications and changes can be made without departing from the scope of the invention as set forth in the claims below. Other examples that occur to those skilled in the art are intended to be within the scope of the claims if they have structural elements that do not differ from the literal language of the claims, or if they include equivalent structural elements with insubstantial differences from the literal languages of the claims. Accordingly, the specification and figures are to be regarded in an illustrative rather than in a restrictive sense, and all such modifications are intended to be included within the scope of the invention.

Benefits, other advantages, and solutions to problems have been described above with respect to one or more specific embodiments. After reading the specification, those of ordinary skill in the art will appreciate that certain features that are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range and include sub-ranges that overlap from one preferred range to another preferred range.

What is claimed is:

1. A syringe, comprising:
a barrel comprising a cylindrical body portion and a nose end;
a needle assembly comprising a needle received within the needle assembly and extending from the nose end of the barrel; and
a plunger assembly comprising a plunger surface for selectively actuating the plunger assembly, an elongated plunger body extending therefrom, and a plunger seal that forms a fluid tight seal between the cylindrical body portion of the barrel and the plunger assembly, wherein the elongated plunger body comprises a ribbed portion, an annular clip channel disposed forwardly of the ribbed portion, and a disabling clip disposed around the elongated plunger body and configured to engage with the cylindrical body portion of the barrel to prevent withdrawal of the plunger assembly from the barrel after first use of the syringe.

2. The syringe of claim 1, wherein the ribbed portion of the elongated plunger body comprises a series of beveled ribs extending along a length of the elongated plunger body of the plunger assembly, and wherein a beveled portion of each of the beveled ribs is angled rearwardly towards the plunger surface of the plunger assembly.

3. The syringe of claim 1, wherein an annular clip stop provides a rearward seating surface for the plunger seal, and wherein the annular clip channel is disposed between the ribbed portion and the annular clip stop.

4. The syringe of claim 1, wherein the disabling clip comprises a main annular body portion disposed around the elongated plunger body and a notch that provides the main annular body portion with a substantially C-shaped cross-sectional profile to enable the main annular body portion of the disabling clip to be laterally pressed onto the elongated plunger body of the plunger assembly.

5. The syringe of claim 4, wherein the notch comprises a width less than a diameter of the elongated plunger body.

6. The syringe of claim 4, wherein the disabling clip comprises a plurality of angled legs extending angularly from the main annular body portion of the disabling clip.

7. The syringe of claim 6, wherein each of the plurality of angled legs comprises one or more barrel tangs extending from an end of each of the plurality of angled legs.

8. The syringe of claim 1, wherein the disabling clip is formed from a corrosion-resistant metal, such as nickel, stainless steel, titanium, or any alloy thereof.

9. The syringe of claim 1, wherein the disabling clip is configured to allow the ribbed portion of the elongated plunger body to slide through the disabling clip while the disabling clip is held longitudinally stationary by an annular stop ridge disposed at a rearward portion of the cylindrical body portion of the barrel during withdrawal of the plunger assembly within the barrel to draw a medicine or drug into the cylindrical body portion of the barrel.

10. The syringe of claim 9, wherein the disabling clip is configured to lock into the annular clip channel of the elongated plunger body of the plunger assembly when the plunger assembly is fully withdrawn within the barrel.

11. The syringe of claim 10, wherein the disabling clip is retained within the annular clip channel and rides forward in the annular clip channel when the plunger assembly is advanced within the barrel to administer an injection with the syringe.

12. The syringe of claim 11, wherein one or more barrel tangs extending from the disabling clip engage with the cylindrical body portion of the barrel to prevent withdrawal of the plunger assembly from the barrel after the first use of the syringe.

13. The syringe of claim 12, wherein engagement between the one or more barrel tangs and the cylindrical body portion of the barrel automatically disables subsequent use of the syringe after the first use of the syringe.

14. The syringe of claim 1, wherein the barrel comprises a pair of opposing indicia display surfaces that extend substantially tangentially from the cylindrical body portion of the barrel, and wherein a needle safety device is slidably engaged with the pair of opposing indicia display surfaces between a first retracted position prior that exposes a needle tip of the needle for selective use of the syringe and a second extended position to cover up the needle tip of the needle after the first use of the syringe.

15. The syringe of claim 14, wherein the barrel comprises a pair of slide stops that engage the pair of channels of the needle safety device and lock the needle safety device in the second extended position to prevent unintended needle sticks or inadvertent contact with the needle after the first use of the syringe.

16. The syringe of claim 1, wherein the needle assembly comprises a needle holder that retains the needle, wherein the needle assembly is rearwardly biased against the nose end of the barrel by a compression spring, and wherein an elastomeric seal is disposed between a head section of the needle holder and the barrel to hold the needle assembly in fixed relation to the barrel prior to retraction of the needle assembly.

17. The syringe of claim 16, wherein the plunger assembly comprises a main plunger body comprising the plunger surface for selectively actuating the plunger assembly and the elongated plunger body extending therefrom, wherein the plunger assembly comprises a needle retraction actuator that engages with the elongated plunger body of the main plunger body, and wherein the needle retraction actuator is driven forwardly with forward motion of the main plunger body.

18. The syringe of claim 17, wherein the needle retraction actuator is configured to contact and move the elastomeric seal forward in the barrel in response to activation of the plunger assembly to disengage the elastomeric seal from the needle holder to initiate retraction of the needle assembly into the barrel, and wherein the needle assembly retracts rearwardly into an inner cavity of the needle retraction actuator.

19. A method of administering an injection, comprising:
providing a syringe comprising: a barrel comprising a cylindrical body portion and a nose end; a needle assembly comprising a needle received within the needle assembly and extending from the nose end of the barrel; and a plunger assembly comprising a plunger surface for selectively actuating the plunger assembly, an elongated plunger body extending therefrom, and a plunger seal that forms a fluid tight seal between the cylindrical body portion of the barrel and the plunger assembly, wherein the elongated plunger body comprises a ribbed portion, an annular clip channel disposed forwardly of the ribbed portion, and a disabling clip disposed around the elongated plunger body;
drawing a medicine through the needle into the barrel by activation of the plunger assembly in a first direction;
injecting the medicine from the barrel through the needle by activation of the plunger assembly in a second opposing direction; and
preventing subsequent use of the syringe via engagement of the disabling clip with the cylindrical body portion of the barrel to prevent withdrawal of the plunger assembly from the barrel after first use of the syringe.

20. The method of claim 19, further comprising: sliding the ribbed portion of the elongated plunger body through the disabling clip while the disabling clip is held longitudinally stationary by an annular stop ridge disposed at a rearward portion of the cylindrical body portion of the barrel during withdrawal of the plunger assembly within the barrel in the first direction to draw a medicine or drug into the cylindrical body portion of the barrel.

21. The method of claim 20, further comprising: retaining the disabling clip within the annular clip channel when the plunger assembly is advanced within the barrel in the second opposing direction to administer an injection with the syringe.

22. The method of claim 21, further comprising: preventing withdrawal of the plunger assembly from the barrel in the first direction and automatically disabling subsequent use of the syringe after the first use of the syringe through engagement of one or more barrel tangs extending from the disabling clip with the cylindrical body portion of the barrel.
